(19) United States
(12) Patent Application Publication
(10) Pub. No.: US 2004/0181104 A1
(43) Pub. Date: Sep. 16, 2004

(54) Catalyst and process for alkyl group dehydrogenation of organic compounds

(75) Inventors: Chuen Y. Yeh, Edison, NJ (US); Zhiping Shan, Bloomfield, NJ (US); Philip J. Angevine, Woodbury, NJ (US); Dinesh Gandhi, Towoco, NJ (US)

Correspondence Address: DILWORTH & BARRESE, LLP, 333 EARLE OVINGTON BLVD., UNIONDALE, NY, 11553, US

(73) Assignee: ABB Lummus Global Inc.

(21) Appl. No.: 10/791,565

(22) Filed: Mar. 2, 2004

Related U.S. Application Data
(60) Provisional application No. 60/453,462, filed on Mar. 10, 2003.

(51) Int. Cl. C07C 4/02; B01J 31/00

(52) U.S. Cl. 585/444; 502/150; 502/152; 502/153; 502/155; 502/158

(57) ABSTRACT

A dehydrogenation catalyst includes an organometallic pincer complex bonded to a mesoporous inorganic oxide support, the organometallic pincer complex possessing catalytic activity for alkyl group dehydrogenation. The pincer complex includes at least one element selected from Group VIII or Group IB of the Periodic Table of the elements, and at least one element selected from Group VA of the Periodic Table of the elements in each of two molecular arms, the Group VIII or Group IB element being bonded to each of the Group VA elements. The catalyst is advantageously employed in conjunction with catalytic distillation to permit the dehydrogenation of organic compounds at lower temperatures and at lower cost than conventional methods.

### CROSS-REFERENCE TO RELATED APPLICATIONS

This application claims priority to U.S. provisional application Serial No. 60/453,462 filed Mar. 10, 2003, to which priority is claimed.

### BACKGROUND

1. Technical Field

The present disclosure relates to a catalyst and process for the catalytic dehydration of saturated alkyl groups in organic compounds to produce corresponding olefinic groups.

2. Background of the Art

Alkanes are abundant, but significantly less reactive than olefins, and cannot be directly used as building blocks for synthesizing other petrochemicals or polymers. Conversion of alkanes into reactive molecules has long been a subject of study for academic and industrial research. One way to convert alkanes into reactive molecules is by the dehydrogenation of the alkane to produce the corresponding olefin, e.g., converting ethane to ethylene, or ethylbenzene to styrene monomer. However, such dehydrogenation reactions are energy intensive and require high temperatures.

An industrially important reaction of this type is the dehydrogenation of ethylbenzene to styrene, which is very endothermic. This process typically is carried out at temperatures of 600° C. to 650° C. in the presence of a large volume of steam to facilitate conversion and selectivity to the desired products. The combination of high energy consumption and the need for high temperature equipment contribute to a high production cost for the currently used routes.

Recent advances in organometallic homogeneous catalysis enable high catalytic activity in alkane dehydrogenation under milder conditions. Particularly useful are the noble metal “pincer” complexes such as IrH₂ {C₆H₃(CH₂PR₂)₂-2,6} wherein R is a tert-butyl or isopropyl group. These organometallic pincer catalysts have shown unusually high activity for alkane dehydrogenation in the liquid phase. See e.g., Craig M. Jensen, Iridium PCP Pincer Complexes: Highly Active and Robust Catalysts fore Novel Homogeneous Aliphatic Dehydrogenations, Chem. Commun. 1999, pgs. 2443-2449; Gupta et al., Catalytic Dehydrogenation of Ethylbenzene and Tetrahydrofuran by a Dihydrido Iridium P-C-P Pincer Complex, Chem. Commun., 1997, pgs. 461-462; and Liu et al., Efficient Thermochemical Alkane Dehydrogenation and Isomerization Catalyzed by an Iridium Pincer Complex, Chem. Commun., 1999, pgs. 655-656, all of the aforementioned articles being incorporated by reference herein. U.S. Pat. No. 5,780,701 to Kaska et al., which is herein incorporated by reference, discloses a pincer type catalyst for the dehydrogenation of alkanes. WO 02085920 discloses the introduction of a second transition metal p-bonded to an η⁵-aromatic ligand, and a pincer ligand.

However, although these homogeneous organometallic pincer complexes have desirable catalytic properties, they nevertheless suffer from practical problems which prevent their commercial use. In particular, complete recovery of these catalysts from the liquid phase is not practicable. Also, since these catalysts employ noble metals, even small losses due to inefficient recovery can render their use uneconomical. Accordingly, there needs to be a way to immobilize these catalysts upon a solid substrate to prevent their loss.

Also, dehydrogenation of alkanes to alkenes is thermodynamically unfavored, especially at low temperatures. Alkane conversion is usually too low to have commercial value. A way to shift the equilibrium towards alkane conversion in this reversible reaction is to instantly remove the reaction products such as hydrogen and alkenes. Moreover, in addition to the undesirable thermodynamic effect of the alkenes upon the reaction, the activity of the catalyst is inhibited by the high concentration of alkenes. Catalyst activity can be reduced because the alkenes can combine with the noble metal in the catalyst to form complexes. Thus, what is needed is a new reactor design which facilitates the immediate separation of the alkenes and other reaction products from the alkane.

### SUMMARY OF THE INVENTION

A method and catalyst for the dehydrogenation of organic compounds are provided herein. The dehydrogenation catalyst comprises an organometallic pincer complex bonded to a mesoporous inorganic oxide support, the organometallic pincer complex possessing catalytic activity for alkyl group dehydrogenation. The pincer complex includes at least one element selected from Group VIII or Group IB of the Periodic Table of the elements, and at least one element selected from Group VA of the Periodic Table of the elements in each of two molecular arms (the details of which are later described), the Group VIII or Group IB element being bonded to each of the Group VA elements. The catalyst is advantageously employed in conjunction with catalytic distillation to permit the dehydrogenation of organic compounds at lower temperatures and at lower cost.

### BRIEF DESCRIPTION OF THE DRAWINGS

Various embodiments are described herein with reference to the drawings wherein:

FIG. 1 is a schematic representation of the process of and equipment of the present invention.

### DETAILED DESCRIPTION OF PREFERRED EMBODIMENT(S)

The invention is useful for the dehydrogenation of alkyl group-containing organic compounds to form the corresponding unsaturated compounds. The organic compound can be any organic molecule possessing an alkyl group according to formula I which can be dehydrogenated to an olefinic group according to formula II:

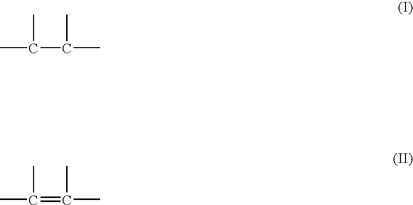

The organic compound can include, for example, non-hydrocarbon compounds such as molecules containing one or more heteroatoms in a ring structure (e.g., cyclic ethers such as tetrahydrofuran) or as substituents on a ring. The organic compound also can include unsaturated or aromatic linkages in the molecular structure. However, the preferred organic compound is a saturated alkane hydrocarbon. As used herein the term “alkane” encompasses straight or branched chain alkyl compounds having the general formula CₙH₂ₙ₊₂ (e.g., ethane, propane, butane, pentane, etc.), saturated cycloaliphatic compounds having the general formula CₙH₂ₙ (e.g., cyclohexane, cycloheptane, cyclooctane, etc.). Many other molecules have a saturated alkyl chain attached to one or more aromatic rings, as well as other functional groups (e.g., —OH, —COOH, —NH₂, etc.). In these molecules the saturated alkyl chain can undergo dehydrogenation. An important example is the dehydrogenation of ethylbenzene to styrene. Another example is the dehydrogenation of methylcyclohexane to toluene.

The catalyst includes an organometallic pincer complex which is immobilized on a solid inorganic oxide support by means of chemical bonding by a bridging molecule. The preferred inorganic oxide has a mesoporous structure. An advantageous feature of the mesoporous inorganic oxide support is that its pore size (2 nm to 50 nm) is optimal with respect to both accessibility and surface. While zeolites are often used in hydrocarbon reactions, their pore size is too small to permit access of the reactant and the bridging molecule. Macropores, on the other hand, suffer from low surface area. The pincer catalyst contains at least one metal component from Group VIII of the Periodic Table of the elements such as iron, cobalt, nickel, or, preferably, a noble metal such as platinum, palladium, iridium, rhodium, ruthenium or osmium, or a Group IB metal such as copper, silver or gold, and a Group VA element such as phosphorus, nitrogen, arsenic or antimony. The pincer complex includes two molecular; “arm” portions extending outwardly from a molecular core. Each arm portion includes a Group VA element. The Group VIII element or Group IB element is bonded directly or indirectly to the core and is positioned between, and bonded to, the Group VA elements in the arm portions. The molecular core can be a ring-containing structure such as a benzene ring or other aromatic ring, a saturated or unsaturated carbocyclic structure, or a straight or branched chain structure. In one embodiment the pincer complex has the molecular structure III below.

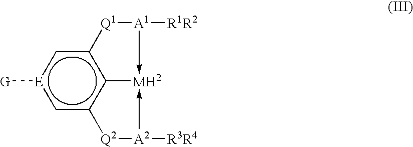

wherein A¹ and A² can be the same or different and are each independently phosphorus, nitrogen, arsenic or antimony. E, which is in a position para to the M, is carbon, silicon or germanium, and can optionally have a substituent group G attached thereto wherein G can be —OH, —NH₂, —SH, —OR⁵, —R⁵C═C, —R⁶OH, —R⁶NH₂, —R⁶COOH, or —R⁶COOR⁷ wherein R⁵ is an alkyl group having from 1 to 10 carbon atoms, R⁶is a substituted alkyl group with Up to 5 carbon atoms, and R⁷ is an alkyl group having from about 1 to 10 carbon atoms. M is a Group VIII metal or Group IB metal, preferably a noble metal selected from platinum, palladium, iridium, rhodium, ruthenium and osmium, Q¹ and Q² can be the same or different moieties and are each independently —CH₂—, —CH₂CH₂—, or —CH═CH—. And R¹ R², R³ and R⁴ can be the same or different moieties and are each independently selected from alkyl, alkenyl, cycloalkyl and aryl groups having from 1 to 10 carbon atoms, or R¹ and R² together and/or R³and R⁴ together form a ring structure having from about 4 to about 10 carbon atoms. As can be seen, the Q¹-A¹-R¹R² group and the Q²-A²-R³R⁴ group constitute the two arm portions of the pincer complex with the Group VIII or Group IB metal M positioned between, and bound to, A¹ and A². A preferred pincer complex has the formula IrH₂{C₆H₂G(CH₂PR₂)₂-2,6} wherein R is a tert-butyl or isopropyl group.

Various methods for making pincer complexes are known to those with skill in the art. For example, in one method, 5-hydroxyisophthalic acid is reacted with dimethyl sulfate, potassium carbonate and acetone to produce 3,5-dicarbomethoxyanisol, which is reacted with lithium aluminum hydride to produce 3,5-di(hydroxymethyl)anisole, which is reacted with phosphorus tribromide in benzene to produce 3,5-di(bromomethyl)anisole, which is reacted with HP(t-butyl)₂ and then NaOAc to produce the pincer ligand 3,5-di(di-t-butylphosphinomethyl)-anisole, which may be reacted with the trihydrate of iridium trichloride, 2-propanol to form an iridium-containing complex. The iridium-containing complex is then reacted with sodium hydride, hydrogen in tetrahydrofuran with sonication and then subjected to a vacuum with the removal of hydrogen to form an iridium-containing pincer complex suitable for use in the present invention.

The inorganic support to which the pincer organometallic complex is bonded is preferably mesoporous and includes such materials as disclosed in U.S. Pat. Nos. 5,098,684, 6,358,486, both of which are incorporated by reference, or those disclosed the literature articles: Zhao et al., Science 1998, Vol. 279, pgs 548-552 and Bagshaw et al., Science, 1995, Vol. 269, pg. 1242, both of which are incorporated by reference herein. Preferably, the mesoporous inorganic oxide support is a three-dimensional mesoporous inorganic oxide material having at least 97 volume percent mesopores based on micropores and mesopores of the inorganic oxide, and having an X-ray diffraction peak at between 0.3 and 3 degree in 2θ, having surface area of 400-1100 m²/g, and having total pore volume of about 0.3-2.2 cm³/g, the mesopores being randomly interconnected. The mesoporous inorganic oxide support and a method for making it are described in U.S. Pat. No. 6,358,486. The average mesopore size of the preferred catalyst support, as determined from N₂-porosimetry, ranges from about 2 nm to about 25 nm.

Generally, the mesoporous inorganic oxide is prepared by heating a mixture of (1) a precursor of the inorganic oxide in water, and (2) an organic templating agent that mixes well with the oxide precursor or the oxide species generated from the precursor, and preferably forms hydrogen bonds with it. The starting material is generally an amorphous material and may be comprised of one or more inorganic oxides such as silicon oxide or aluminum oxide, with or without additional metal oxides. The silicon atoms may be replaced in part by metal atoms such as aluminum, titanium, vanadium, zirconium, gallium, manganese, zinc, chromium, molybdenum, nickel, tin, cobalt and iron and the like. The additional metals may optionally be incorporated into the material prior to initiating the process for producing a structure that contains mesopores. Also after preparation of the material, cations in the system may optionally be replaced with other ions such as those of an alkali metal (e.g., sodium, potassium, lithium, etc.).

The inorganic oxide precursor can be amorphous silica such as silica gel or a silicate such as tetraethyl orthosilicate (TEOS) or a source of aluminum such as aluminum oxide, aluminum hydroxide or aluminum isopropoxide. TEOS, silica gel. aluminum oxide, aluminum hydroxide and aluminum isopropoxide are commercially available from known suppliers. The organic templating agent is preferably a glycol (a compound that includes two or more hydroxyl groups), such as glycerol, diethylene glycol, triethylene glycol, tetraethylene glycol, propylene glycol, and the like, or member(s) of the group consisting of triethanolamine, triisopropanolamine, sulfolane, tetraethylene pentamine and diethylglycol dibenzoate.

The mesoporous catalyst support is a pseudo-crystalline material. The diameter of the mesopores is preferably from about 2 nm to about 25 nm. The surface area of the catalyst support, as determined by BET (N₂), preferably ranges from about 400 m²/g to about 1200 m²g. The catalyst support pore volume preferably ranges from about 0.3 cm³/g to about 2.2 cm³/g. The catalyst support can further comprise metals selected from Groups IB, IIB, IIIB, IVB, VB, VIB, VIIB VIII, IVA and VA of the Periodic Table of the Elements.

A “bridging molecule” is used to connect the pincer complex to the catalyst support. Bridging molecules can include, for example, 3-(triethoxysilyl) propylisocyanate and 3-(triethoxysilyl) chloropropane. The functional group at the end of the bridging molecule (e.g., isocyanate, chloro) can be reacted with the para-substituent G at E of the pincer complex molecule to link the bridging molecule to the pincer complex. Methods for conducting this reaction are known to those with skill in the art. The triethoxysilyl groups at the other end of the bridging molecule can be hydrolyzed for chemically bonding to the mesoporous support via silanol condensation.

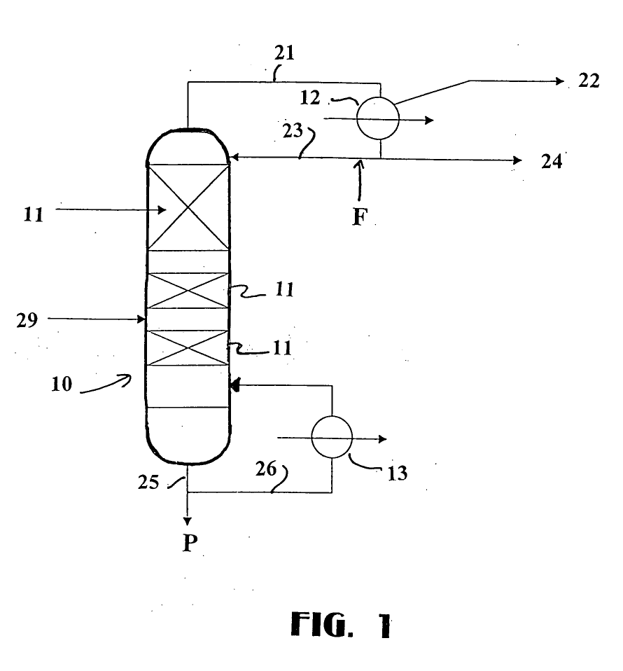

Referring now to FIG. 1, a system and method are shown for the catalytic dehydrogenation of ethylbenzene to produce styrene. A catalytic distillation column 10 includes one or more packed beds 11 of the catalyst described herein containing a pincer complex bonded to a mesoporous support. A feed F containing ethylbenzene is preferably introduced into the recycle stream 23. The dehydrogenation reaction is conducted in the liquid phase at a temperature of from about 100° C. to about 150° C. and a pressure of from about 0.01 atmospheres to about 1 atmosphere as the ethylbenzene flows downward through the catalyst beds 11. For ethylbenzene dehydrogenation the primary reaction is as follows:

ethylbenzene→styrene+hydrogen

The overhead stream 21 containing hydrogen and unreacted ethylbenzene, along with other aromatic components (impurities such as benzene and toluene) is removed and condensed by chiller 12. Uncondensed hydrogen is drawn off at line 22. The remaining liquid stream is divided with one portion being recycled to the catalytic distillation column 23 and the other portion being drawn off for benzene and toluene removal. It is necessary to remove a portion of the recycle stream to prevent buildup and unnecessary recycling of benzene and toluene. The hydrogen is removed from the reaction as soon as it is formed, thereby shifting the reaction towards the production of styrene. In one embodiment the hydrogen is removed by physical separation. Alternatively, the hydrogen can be scavenged by introducing a sacrificial molecule, such as an alkene (e.g., t-butylethene) into the reaction zone to scavenge the hydrogen by a hydrogenation reaction. The selected alkene is preferentially hydrogenated rather than the styrene, and is converted to the corresponding alkane. Optionally, the alkene can be introduced into the feed.

The styrene is withdrawn from the bottom of the reactor. Part of the styrene stream is recycled via line 26 and passes through the reboiler 13 as heat input to the catalytic distillation reactor. Another portion of the styrene is drawn off as product stream P. A polymerization inhibitor, preferably 2-sec-butyl-4,6-nitrophenol (“DNBP”), is injected into the column 10 via line 29 to prevent premature polymerization of the styrene. Other polymerization inhibitors are known and include, for example, 4-tert-butyl catechol (“TBC”), which is preferably added to the product styrene.

Another important application of the present invention is the dehydrogenation of linear alkanes having up to about 40 carbon atoms to produce corresponding 1-alkenes, which are useful for the synthesis of oxo alcohols and surfactants.

The example below illustrates features of the invention.

### EXAMPLE 1

This example illustrates the synthesis of a mesoporous inorganic oxide support. First, 20.32 parts tetraethylorthosilicate (TEOS) were added to 16.32 parts water and stirred. After continuous stirring for 30 minutes, 9.33 parts triethanolamine were added. After stirring again for another 30 minutes, 4.02 parts tetraethylammonium hydroxide aqueous solution (35% solution available from Aldrich) were added drop-wise to the mixture to increase the pH. After stirring for about 2 hours, the mixture formed a thick non-flowing gel. This gel was aged at room temperature under static conditions for 17 hours. Next, the gel was dried in air at 100° C. for 28 hours. The dried gel was transferred into an autoclave and hydrothermally treated at 170° C. for 17.5 hours. Finally, it was calcined at 600° C. for 10 hours in air with a heating ramp rate of 1° C./min. The final product was a meso-structured material.

While the above description contains many specifics, these specifics should not be construed as limitations on the scope of the invention, but merely as exemplifications of preferred embodiments thereof. Those skilled in the art will envision many other possible variations that are within the scope and spirit of the invention as defined by the claims appended hereto.

What is claimed is:

1. A dehydrogenation catalyst which comprises:
an organometallic pincer complex bonded to an inorganic oxide support, said organometallic pincer complex possessing catalytic activity for the dehydrogenation of alkyl groups.

2. The dehydrogenation catalyst of claim 1 wherein the pincer complex includes at least one element selected from Group VIII or Group IB of the Periodic Table of the elements, and at least one element selected from Group VA of the Periodic Table of the elements in each of first and second molecular arm portions, the Group VIII or Group IB element being bonded to each of the Group VA elements.

3. The dehydrogenation catalyst of claim 2 wherein the first and second molecular arm portions are each bonded to a molecular core portion, the Group VIII or Group IB element being bonded directly or indirectly to the molecular core portion.

4. The dehydrogenation catalyst of claim 3 wherein the molecular core portion comprises an aromatic ring.

5. The dehydrogenation catalyst of claim 4 wherein the first molecular arm portion comprises a Q¹-A¹-R¹R² group and the second molecular arm portion comprises a Q²-A²-R³R⁴ group, wherein A¹ and A² are the same or different and are each independently selected from phosphorus, nitrogen, arsenic and antimony, Q¹ and Q² are the same or different and are each independently selected from —CH₂—, —CH₂CH₂—, and —CH═CH—, and R¹ R², R³ and R⁴ are the same or different and are each independently selected from alkyl, alkenyl, cycloalkyl and aryl having from 1 to 10 carbon atoms, or R¹ and R² together and/or R³and R⁴ together form a ring structure having from about 4 to about 10 carbon atoms.

6. The dehydrogenation catalyst of claim 1 wherein the pincer complex has the formula: 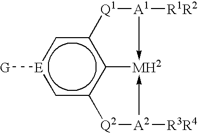
wherein A¹ and A² can be the same or different and are each independently phosphorus, nitrogen, arsenic or antimony, E is carbon, silicon or germanium, G is optional and is selected from the group consisting of —OH, —NH₂, —SH, —OR⁵, —R⁵C═C, —R⁶OH, —R⁶NH₂, —R⁶COOH, or —R⁶COOR⁷ wherein R⁵ is an alkyl group having from 1 to 10 carbon atoms, R⁶ is a substituted alkyl group with up to 5 carbon atoms, and R⁷ is an alkyl group having from about 1 to 10 carbon atoms, M is a Group VIII or Group IB metal, Q¹ and Q² can be the same or different and are each independently —CH₂—, —CH₂CH²—, and —CH═CH—, and R¹ R², R³ and R⁴ can be the same or different and are each independently selected from alkyl, alkenyl, cycloalkyl and aryl having from 1 to 10 carbon atoms, or R¹ and R² together and/or R³and R⁴ together form a ring structure having from about 4 to about 10 carbon atoms.

7. The dehydrogenation catalyst of claim 1 wherein the pincer complex has the formula IrH₂{C₆H₂G(CH₂PR₂)₂-2,6} wherein R is a tert-butyl or isopropyl group and G is —OH, —NH₂, —SH, —OR⁵, —R⁵C═C, —R⁶OH, —R⁶NH₂, —R⁶COOH, or —R⁶COOR⁷ wherein R⁵ is an alkyl group having from 1 to 10 carbon atoms, R⁶ is a substituted alkyl group with up to 5 carbon atoms, and R⁷ is an alkyl group having from about 1 to 10 carbon atoms.

8. The dehydrogenation catalyst of claim 1 wherein the pincer complex is bonded to the inorganic oxide support by means of a bridging group.

9. The dehydrogenation catalyst of claim 8 wherein the bridging group is derived from compounds containing a triethoxysilyl group and isocyanate group, or compounds containing a triethoxysilyl group and a halogenated alkane.

10. The dehydrogenation catalyst of claim 8 wherein the inorganic oxide support is a mesoporous inorganic oxide.

11. The dehydrogenation catalyst of claim 1 wherein the inorganic oxide support is a porous inorganic oxide having at least 97 volume percent mesopores based on micropores and mesopores of the inorganic oxide, and having an X-ray diffraction peak at between 0.3 and 3 degree in 2θ, having surface area of 400-1100 m²/g, and having total pore volume of about 0.3-2.2 cm³/g, said mesopores being randomly interconnected.

12. A method for dehydrogenating an organic compound comprising the steps of:
a) providing a reaction zone containing a dehydrogenation catalyst including an organometallic pincer complex bonded to an inorganic oxide support said organometallic pincer complex possessing catalytic activity for alkyl group dehydrogenation;
b) contacting an organic compound possessing at least one alkyl group with said catalyst under dehydrogenation conditions to produce a dehydrogenated organic compound wherein hydrogen is separated from the dehydrogenated compound within the reaction zone.

13. The method of claim 12 wherein the inorganic oxide support is a mesoporous inorganic oxide.

14. The method of claim 12 wherein the organic compound is ethylbenzene and the dehydrogenated compound is styrene.

15. The method of claim 12 wherein the organic compound is an alkane or mixture of alkanes and the dehydrogenated compound is an alkene or mixture of alkenes.

16. The method of claim 12 wherein the organic compound is a cycloalkane or mixture of cycloalkanes and the dehydrogenated compound is a cycloalkene or mixture of cycloalkenes.

17. The method of claim 12 wherein the organic compound is methylcyclohexane and the product is toluene.

18. The method of claim 12 wherein the reaction temperature ranges from about 100° C. to about 150° C.

19. The method of claim 12 further including the step of adding at least one polymerization inhibitor to the styrene.

20. The method of claim 13 wherein the mesoporous inorganic oxide support is a porous inorganic oxide having at least 97 volume percent mesopores based on micropores and mesopores of the inorganic oxide, and having an X-ray diffraction peak at between 0.3 and 3 degree in 2θ, having surface area of 400-1100 m²/g, and having total pore volume of about 0.3-2.2 cm³/g, said mesopores being randomly interconnected.

21. The method of claim 12 wherein the reaction zone is within a catalytic distillation system.

22. A method for dehydrogenating an organic compound comprising the steps of:
a) providing a reaction zone containing a dehydrogenation catalyst including an organometallic pincer complex bonded to a mesoporous inorganic oxide support, said organometallic pincer complex possessing catalytic activity for the dehydrogenation of alkyl groups;
b) contacting an organic compound possessing at least one alkyl group with said catalyst under dehydrogenation conditions to produce a dehydrogenated organic compound wherein the produced hydrogen is removed by reaction with a scavenger molecule.

23. The method of claim 22 wherein the scavenger molecule is an alkene, the alkene being converted by reaction with hydrogen to a corresponding alkane.
